(19) United States
(12) Patent Application Publication
(10) Pub. No.: US 2002/0058922 A1
(43) Pub. Date: May 16, 2002

(54) Sanitary garment

(76) Inventor: Terje Skog, Nykirke (NO)

Correspondence Address: YOUNG & THOMPSON, 745 SOUTH 23RD STREET 2ND FLOOR, ARLINGTON, VA, 22202

(21) Appl. No.: 09/982,910

(22) Filed: Oct. 22, 2001

Related U.S. Application Data
(60) Provisional application No. 60/241,800, filed on Oct. 20, 2000.

(51) Int. Cl. A61F 13/15; A61F 13/20

(52) U.S. Cl. 604/389; 604/390; 604/385.01

(57) ABSTRACT

The invention relates to a sanitary garment (1) for a diaper, an incontinence garment or the like, comprising a casing (3, 4), an outer side of the casing being distal from the body of the wearer in use of the garment and an inner side being proximal to the body of the wearer in use of the garment, said casing having a front part (5), a rear part (6) and a crotch part (7) lying between the front and rear parts, and fastening tabs (11, 12) laterally projecting from each side of the casing in the rear or front part thereof, said fastening tabs (11, 12) being portions of a band (9) which is laterally extending over the width of the casing. According to the invention the band (9) is extended over the inner side of the garment and attached to the casing (3, 4) in side portions (11, 12) thereof but essentially unattached to the casing in a central portion (10) thereof, the band (9) being elastic in the side portions (11, 12) attached to the casing but non-elastic in the central portion (10), the attachment of the band to the casing being made with the band extended from a relaxed state.

### TECHNICAL FIELD

The present invention relates to a sanitary garment for a diaper, an incontinence garment or the like, comprising a casing, an outer side of the casing being distal from the body of the wearer in use of the garment and an inner side being proximal to the body of the wearer in use of the garment, said casing having a front part, a rear part and a crotch part lying between the front and rear parts, and fastening tabs laterally projecting from each side of the casing in the rear or front part thereof, said fastening tabs being portions of a band which is laterally extending over the width of the casing.

### BACKGROUND OF THE INVENTION

Sanitary garments, such as diapers, incontinence garments and the like, are often provided with waist elastics in order to enhance the fit of the garment on a wearer. One way of incorporating waist elastics into a sanitary garment is to attach laterally extended elastic threads in a stretched condition between the backing sheet and the top sheet of the garment. In such a garment a lot of folds are created in the casing sheets when the elastic threads are allowed to retract from the stretched condition to a relaxed condition. However, in the central portion of the waist the non-elastic absorbent body restricts the retraction of the elastic threads, thereby preventing full use of the elasticity of the threads and also causing residual stresses in the casing material and on the absorbent body. The same disadvantages will be obtained by use of a casing of elastic material. Another way of imparting elasticity to the waist of a sanitary garment is to provide the garment with elastic fastening tabs anchored to the side portions of the rear or front part of the garment. If such tabs are attached to the outer side of the garment it can be difficult to get hold of the tabs for a person applying the garment to a child lying on his/her back. If such tabs are attached to the inner side of the garment the anchored ends of the tabs can come into contact with the skin of the wearer causing irritation thereof.

In the applied condition of diapers, incontinence garments and the like considerable lateral forces act on the casing material along the waist line thereof. The casing material will also be influenced by longitudinally directed forces due to the weight of the absorbent body. Since many materials used as casing material for sanitary garments have a greater strength in a machine direction, which preferably is a direction substantially parallel to the longitudinal direction of the garment, than in a direction perpendicular thereto it would be advantageous to eliminate or at least greatly reduce the lateral forces acting on the casing material in the waist area of the garment. In WO 99/20214 is disclosed a diaper having fastening tabs that are part of a band disposed between the top sheet and a backing sheet and fastened to at least the backing sheet in the waist area of the rear part of the diaper. The band is non-elastic and the lateral forces acting on the back part of the backing sheet is greatly reduced.

An object of the present invention is to provide a sanitary garment having elastic fastening tabs without the drawbacks referred to above, in which the lateral forces acting on the rear waist area of the casing is reduced.

Another object is to provide a sanitary garment with a pocket for receiving urine and faeces or for firmly holding the end part of an absorbent insert.

### SUMMARY OF THE INVENTION

These objects are achieved by a sanitary garment for a diaper, an incontinence garment or the like, comprising a casing, an outer side of the casing being distal from the body of the wearer in use of the garment and an inner side being proximal to the body of the wearer in use of the garment, said casing having a front part, a rear part and a crotch part lying between the front and rear parts, and fastening tabs laterally projecting from each side of the casing in the rear or front part thereof, said fastening tabs being portions of a band which is laterally extending over the width of the casing, characterised in that the band is extended over the inner side of the garment and attached to the casing in side portions thereof but essentially unattached to the casing in a central portion thereof, the band being elastic in the side portions thereof attached to the casing but non-elastic in the central portion, the attachment of the band to the casing being made with the band extended from a relaxed condition. In such an article the casing can be made without waist elastics, the waist elastics being provided solely by the elastic portions of the band, the manufacture of the article thereby being simplified. Since the band runs continuously around the waist portion of the rear or front part of the casing on the inner side thereof it is easy to attach to the casing without risk for sharp edges thereof coming in to contact with the skin of a wearer during use. Moreover, all lateral forces in the waist portion of the rear or front part of the article will be taken up by the band, the casing material can thereby be chosen without consideration of such forces. The placing of the band on the inner side of the casing enables the outer side to be given an attractive aesthetic appearance unbroken by components attached thereto.

In a preferred embodiment the band consists of non-woven material, to which an elastic film is laminated in the side portions of the band. The fastening tabs comprise fastening elements adapted to engage complementary fastening elements on the outer side of the casing in the part thereof being opposite to the part containing the band, said complementary fastening elements preferably being disposed on a single strip of material, thereby taking up all lateral load in this part of the garment. Advantageously, the fastening elements consist of a hooks and loops type of fastener. Preferably, the garment includes leg elastics.

The outer edge of the central portion of the band could in the preferred embodiment be fastened to the top sheet by a narrow join and at least the central portion of the band can be made of liquid-impermeable material. In such a case the central portion of the band functions as a closed pocket for receiving and holding urine or faeces flowing on the inner side of the top sheet and also as a sealing element between the back of the wearer and the top sheet.

The sanitary garment can have an absorbent insert disposed on the casing, one end part of the insert being inserted between the casing and the central portion of the band, the other end part being inserted into a pocket provided on the part of the casing being opposite to the part containing the band.

In the preferred embodiment the casing has laterally extending side portions in the front and rear parts thereof, the width of the band essentially corresponding to the longitudinal extension of these side portions.

### BRIEF DESCRIPTION OF THE DRAWING

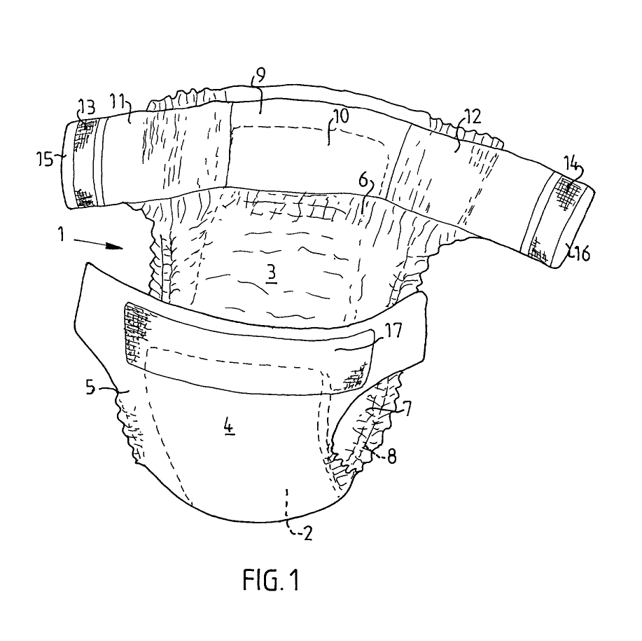

The invention will now be described with reference to FIG. 1 which schematically shows a preferred embodiment of the invention in a perspective view.

### DESCRIPTION OF EMBODIMENTS OF THE INVENTION

In FIG. 1 a diaper 1 is disclosed. As is conventional in this field of the art, the diaper comprises an absorbent body 2, being enclosed between a liquid-permeable top sheet 3 and a liquid-impermeable backing sheet 4, these sheets being fastened to each other in portions thereof reaching beyond the absorbent body. The diaper has a front part 5, a rear part 6 and an intermediate crotch part 7, the longitudinal direction of the diaper extends from the rear to the front edge thereof. The diaper also comprises leg elastics 8, for example one or several elastic threads or bands disposed between the top sheet and the backing sheet along the sides of the diaper, at least in the crotch part thereof, and attached to the top sheet and/or backing sheet in a stretched state. The top sheet 3 and the backing sheet 4 constitutes a casing for the absorbent body 2 and the side portions of the front 5 and rear part 6 of the diaper 1 are laterally projecting in relation to the extension of the casing in the crotch part 7 of the diaper.

According to the invention, a band 9 extends laterally over the rear part of the diaper and projects outwardly from the sides thereof. The band 9 comprises a non-elastic central portion 10 and two elastic side portions 11, 12. The band 9 is attached to the top sheet 3 only in the elastic side portions and fastened thereto in a stretched state by welding or gluing. When the elastic side portions 11, 12 are allowed to relax a lot of folds will be created in the side portions of the top sheet and the backing sheet, which is attached to the top sheet in portions outside the absorbent body 2, as described above. In the end portions of the laterally projecting portions of the band 9, fastening elements 13, 14 for fastening the side portions of the rear and front part of the diaper to each other in order to give the diaper a pants-like configuration, are attached to the inner side of the band. Preferably, these fastening elements are distanced from the respective end of the band in order to provide grip tabs 15, 16 for the user of the diaper. Such grip tabs will typically have an extension of about 3-17 mm in the lateral direction.

The fastening elements 13, 14 are in the shown embodiment male fastener elements in fasteners of the hooks and loops type. Other types of mechanical fasteners can of course also be used, such as snap fasteners, buttons and holes and the like. It is also conceivable to use adhesive fasteners.

The elastic side portions 11, 12 of the band 9 need not be elastic over their entire length, i.e. their extension in a lateral direction, but can have non-elastic parts. It is for example preferred to have the parts of portions 11, 12 to which the fastening elements 13, 14 are attached as well as the grip tabs 15, 16 made of non-elastic material. In this respect it is pointed out that by non-elastic materials is meant materials that can withstand the forces a diaper normally is subjected to during manufacturing, packaging and use without any appreciable elongation or deformation. The fastening elements 13, 14 co-act with a strip 17 of material provided with female fastening elements, which is laterally extending over the waist area of the front part 5 of the diaper. In the shown embodiment, the strip 17 is made of a loops-type material suitable to be engaged by the hooks on the fastening elements 13, 14. However, if other types of fasteners are used, female elements need only be provided on certain areas thereof enabling the fastening of the male elements to the strip. Preferably, these areas are larger than the male elements in order to give a further possibility to adjust the length of the waistline of the diaper when applied on a wearer than is admitted by different extension of the side portions of the band 9 and thereby of the folds in the side portions of the rear part of the diaper fastened to these portions. The extension of the strip 17 in the side portions of the front part 5 of the diaper is such that if the male fastening elements 13, 14 are attached to the respective outer end of the strip 17, the opposing side portions of the rear and front parts of the diaper are slightly overlapping.

The liquid-permeable top sheet 3 is made of a soft skin-friendly material. Examples of suitable materials are different types of non-woven. Other material that can be used are perforated films, plastic nets or knitted, crotcheted or woven textile materials or combinations and laminates of the above mentioned types of material. The non-woven material can be formed of natural fibres, such as cellulose or cotton fibres, but can preferably consist of synthetic fibres, such as polyethylene (PE), polypropylene (PP), a polyester, polylactide or regenerated cellulose, or a mix of different fibres. All materials used for top sheets in absorbent sanitary garments can be used for the top sheet 3 and the above mentioned materials are only given as examples.

The liquid-impermeable backing sheet 4 consists of a flexible material, preferably a thin plastic film of PE, PP or a polyester but could also consist of a laminate of a liquid-permeable material, such as a non-woven, and a liquid-impermeable material. All materials that are used for liquid-impermeable backing sheets are conceivable. The backing sheet can advantageously be air-permeable.

The absorbent body 2 is preferably formed of cellulose fibres but also other natural materials, such as cotton fibres or peat can be used. Alternatively can absorbent synthetic fibres or a mixture of natural and synthetic fibres be used. The absorbent body can also comprise a super-absorbent, i.e. a polymer having the capacity to absorb liquid to an amount several times larger than its own weight. The absorbent body can also contain form stabilising and/or liquid dispersing components and also bonding agents for holding together short fibres and particles to a continuous unit. Furthermore, the absorbent body can contain more than one layer of absorbent material, e.g. an acquisition layer on the side proximal to the top sheet. Such an acquisition layer can for example consist of fibre wadding or nonwoven.

The band 9 is preferably made of two non-woven layers, between which strips of elastic material or several elastic threads have been attached in a stretched state in the side portions 11, 12 thereof. Examples of elastic bands that can be used are given in EP-A-494 941. In order to make the band air permeable an air-permeable elastic film, for example made of CaCO₃, can be used. However, the side portions and the central portion of the band 9 can be separate parts joined together in a suitable way, for example by weld and/or glue patterns. Preferably, the inner side of the band which come into contact with the skin of the user is provided with a soft material, such as a non-woven, if the elastic components are not made of a skin-friendly material. Other materials, such as textiles, could be used instead of non-woven for providing a skin-friendly surface on the inner side of the band. The band 9 has preferably a width corresponding to the longitudinal extension of the side portions of the rear part 6 of the diaper 1. However, bands being narrower or wider than the longitudinal extension of the side portions of the rear part can also be used.

The lateral forces to which the diaper is subjected to during use will in the waist area be taken up by the band 9 and the strip 17 and the casing will therefore not be subjected to lateral forces in the waist area. Since the lateral forces to which the diaper can be subjected to during use are small, the casing material can be chosen without consideration of lateral forces. Many of the materials used as casing material for sanitary garments have a greater strength in a machine direction than in a direction perpendicular thereto which means that thinner materials for the casing can be chosen than would have been possible if lateral forces in the waist area have to be considered. Since the lateral forces are taken up by the band 9 and the strip 17 it is possible to use casing materials that are not so strong in the lateral direction. The lateral strength could be defined by the force level up to which the casing material resist lateral forces. Depending on the choice of casing material this level could suitably be from F=1, where F is the lateral force (N/25 mm) of 10% elongation according to the method ASTM D882. The thickness of an air-permeably material has normally a direct influence on the impermeability thereof, the thinner the material is, the higher is the air-permeability. The choice of a thinner material will thus lead to a more comfortable diaper.

When waist elastics is present in the central portion of the waist area, the absorbent body has to be disposed some distance away from the waist line in order not to inhibit the action of the waist elastics. By placing the waist elastics only in the side portions of the diaper 1, the longitudinal extension of the absorbent body in the rear part of the diaper can be chosen without restriction of waist elastics.

Waist elastic is often provided by fastening several laterally extending elastic threads between the top sheet and the backing sheet in the waist area of the casing. However, such a way of manufacturing waist elastic would be quite complicated if the waist elastic should be present only in the side portions. By providing the waist elastics by the side portions 11, 12 of the elastic band 9, the manufacture of the waist elastic is thus facilitated.

The band 9 provides along the length thereof an inner surface of soft non-woven material, which in use of the diaper is in contact with the skin of the wearer. No sharp edges which could chafe against the skin of the user, will thus result from the placing of the band 9 on the top sheet 3. Furthermore, the folds created in the casing material when the band 9 is allowed to relax will not come in contact with the skin of the wearer during use of the diaper. The band 9 can be designed so that in the relaxed or partly relaxed state of the band, the inner surface thereof will be even or the possible folds created have a round appearance without sharp edges. The disposal of the band 9 on the inner side of the casing instead of on the outer side thereof will thus lead to an increase in comfort for the wearer of the diaper.

Furthermore, by giving the band a width essentially corresponding to the longitudinal extension of the laterally projecting side portions of the rear part 6 of the diaper 1 it is ensured that the diaper in the applied state has an unbroken waist line with no loose portions. This gives the diaper a neat appearance and ensures that chafing against loose outer portions of the diaper will not occur during use.

In an preferred embodiment, the rear edge of the central portion of the band can be sealed against the top sheet to provide a sealing against leakage rearwards when the wearer of the diaper is lying on her/his back. In such an alternative embodiment the pocket created by the central portion of the band will be closed in its rear end, i.e the longitudinal edge of the band proximal to the rear edge of the diaper. The band will in such a case preferably consist of liquid impermeable material, at least in the central part thereof, and the central part of the band will provide a pocket for receiving and holding urine and faeces, thereby improving the security against leakage rearwards.

In another alternative embodiment, the casing can be a reusable casing without absorbent body. The absorbent body will in such a case consist of a disposable absorbent body which in use is disposed in the pocket defined by the central portion of the casing, the front part of the casing preferably also being provided with a pocket. In such a case, the casing can be formed of a single or several layer of casing material of the type commonly used for reusable casings for absorbent bodies, such as a textile material. In the event the absorbent body to be used together with such a casing has a layer of liquid-impermeable material turned against the casing, the casing can even be made of liquid permeable material. A reusable casing is in many cases preferred when the invention is used on incontinence garments for adults.

The described embodiments can be modified in several respects without leaving the scope of the invention. For example, the absorbent body can have a shape different from the rectangular shape disclosed in FIG. 1, for example an hour-glass form. Furthermore, the casing can be provided with barrier flaps for preventing lateral leakage and other types of leg elastics than the threads described, for example elastic bands attached to the top sheet along the outer edges thereof by a narrow join. Moreover, the band 9 can be disposed on the front part of the diaper instead of the rear part, the strip 17 then being provided on the rear part. The band 9 need not have a width corresponding to the longitudinal extension of the side portions of the diaper even if this is preferred. The side portions of the band need not have the same width as the central portion. The strip 17 can be substituted by two strips symmetrically disposed in relation to the longitudinal symmetry line of the garment, but this is not preferred. The scope of invention shall therefore be determined solely by the content of the enclosed patent claims.

1. A sanitary garment (1) for a diaper, an incontinence garment or the like, comprising a casing (3, 4), an outer side of the casing being distal from the body of the wearer in use of the garment and an inner side being proximal to the body of the wearer in use of the garment, said casing having a front part (5), a rear part (6) and a crotch part (7) lying between the front and rear parts, and fastening tabs (11, 12) laterally projecting from each side of the casing in the rear or front part thereof, said fastening tabs (11, 12) being portions of a band (9) which is laterally extending over the width of the casing, characterised in that the band (9) is extended over the inner side of the garment and attached to the casing (3, 4) in side portions (11, 12) thereof but essentially unattached to the casing in a central portion (10) thereof, the band (9) being elastic in the side portions (11, 12) attached to the casing but non-elastic in the central portion (10), the attachment of the band to the casing being made with the band extended from a relaxed state.

2. The sanitary garment according to claim 1, characterised in that the band (9) consists of non-woven material, to which an elastic film is laminated in the side portions (11, 12) of the band.

3. The sanitary garment according to claim 1 or 2, characterised in that the fastening tabs (11, 12) comprise fastening elements (13, 14) adapted to engage complementary fastening elements (17) on the outer side of the casing in the part (5) thereof being opposite to the part (6) containing the band

4. The sanitary garment according to claim 3, characterised in that the complementary fastening elements on the outer side of the casing are disposed on a single strip of material (17).

5. The sanitary garment according to claim 3 or 4, characterised in that the fastening elements (13, 14, 17) consist of a hooks and loops type of fastener.

6. The sanitary garment according to any one of claims 1-5, characterised in that the garment includes leg elastics (8).

7. The sanitary garment according to any one of claims 1-6, characterised in that the band (9) is disposed in the rear part (6) and that the rear edge of the central portion (10) of the band is fastened to the inner side of the casing (3, 4) by a narrow join.

8. The sanitary garment according to claim 7, characterised in that at least the central portion of the band is made of liquid-impermeable material.

9. The sanitary garment according to any one of claims 1-8, characterised in that the casing has laterally extending side portions in the front (5) and rear (6) parts thereof, the width of the band (9) essentially corresponding to the longitudinal extension of these side portions.

10. The sanitary garment according to any one of claims 1-9, characterised in that an absorbent insert is disposed on the casing, one end part of the insert being inserted between the casing and the central portion of the band, the other end part being inserted into a pocket provided on the part of the casing being opposite to the part containing the band.
